(19) United States
(12) Patent Application Publication
(10) Pub. No.: US 2005/0079159 A1
(43) Pub. Date: Apr. 14, 2005

(54) In vivo bioreactors

(75) Inventors: Venkatram Prasad Shastri, Philadelphia, PA (US); Molly M Stevens, Drymen (GB); Robert S Langer, Newton, MA (US)

Correspondence Address: FISH & NEAVE IP GROUP;ROPES & GRAY LLP, ONE INTERNATIONAL PLACE, BOSTON, MA, 02110-2624, US

(73) Assignee: Massachusetts Instiute of Technology, Cambridge, MA (US)

(21) Appl. No.: 10/480,682

(22) PCT Filed: Jun. 13, 2002
(86) PCT No.: PCT/US02/18879

Related U.S. Application Data
(60) Provisional application No. 60/297,951, filed on Jun. 13, 2001.

(51) Int. Cl. A61K 45/00

(52) U.S. Cl. 424/93.7

(57) ABSTRACT

The present invention relates to an in vivo method of promoting the growth of autologous tissue and its use to form corrective structures, including tissue that can be explanted to other locations in the animal. In particular, the invention relates to methods and systems for (a) the site-specific regeneration of tissue, and (b) the synthesis of neotissue for transplantation.

### BACKGROUND OF THE INVENTION

Cell differentiation is the central characteristic of morphogenesis which initiates in the embryo, and continues to various degrees throughout the life of an organism in adult tissue repair and regeneration mechanisms. The degree of morphogenesis in adult tissue varies among different tissues and is related, among other things, to the degree of cell turnover in a given tissue. On this basis, tissues can be divided into three broad categories: (1) tissues with static cell populations such as nerve and skeletal muscle where there is no cell division and most of the cells formed during early development persist throughout adult life; (2) tissues containing conditionally renewing populations such as liver where there is generally little cell division but, in response to an appropriate stimulus, cells can divide to produce daughters of the same differentially defined type; and (3) tissues with permanently renewing populations including blood, testes and stratified squamous epithelia which are characterized by rapid and continuous cell turnover in the adult. Here, the terminally differentiated cells have a relatively short life span and are replaced through proliferation of a distinct subpopulation of cells, known as stem or progenitor cells.

Tissue engineering has emerged as a scientific field which has the potential to aid in human therapy by producing anatomic tissues and organs for the purpose of reconstructive surgery and transplantation. It combines the scientific fields of materials science, cell and molecular biology, and medicine to yield new devices for replacement, repair, and reconstruction of tissues and structures within the body. Many approaches have been advocated over the last decade. One approach is to combine tissue specific cells with open porous polymer scaffolds which can then be implanted. Large numbers of cells can be added to the polymer device in cell culture and maintained by diffusion. After implantation, vascular ingrowth occurs, the cells remodel, and a new stable tissue is formed as the polymer degrades by hydrolysis.

A number of approaches have been described for fabricating tissue regeneration devices for either in vitro or in vivo growth of cells. Polymeric devices have been described for replacing organ function or providing structural support. Such methods have been reported by Vacanti, et al., Arch. Surg. 123:545-49 (1988); U.S. Pat. No. 4,060,081 to Yannas, et al.; U.S. Pat. No. 4,485,097 to Bell; and U.S. Pat. No. 4,520,821 to Schmidt, et al. In general, the methods used by Vacanti, et al., and Schmidt, et al., can be practiced by selecting and adapting existing polymer fiber compositions for implantation and seeding with cells, while the methods of Yannas and Bell produce very specific modified collagen sponge-like structures.

However, in most instances, the prior art requires the use of allogeneic transplants, e.g., cells which have at least one MHC mismatch between the donor and recipient. As a consequence, such transplants can be problematic to commercialization as a result of the potential of immuno-rejection of the graft, and/or graft-versus-host response where the graft includes lymphocytes. Accordingly, there is a need for sources of autologous cells for transplantation.

### SUMMARY OF THE INVENTION

One aspect of the present invention relates to a method for promoting generation of soft tissue, or precursor cells for soft tissue, comprising the steps of:
i. creating an artificial space or environment in an organ or cavity of an animal, such as a mammal, and preferably a human; and
ii. introducing into the artificial space or environment a matrix, preferably a dimensionally stable matrix, which is conducive to infiltration by, and growth and/or differentiation of pluripotent cells from the tissue surrounding the artificial space.

In certain preferred embodiments, the artificial space is created adjacent or in periosteum tissue. For instance, the present invention provides a method for promoting generation of cartilage or bone tissue, comprising the steps of:
i. creating an artificial space adjacent or in periosteum tissue of an animal; and
ii. introducing into the artificial space a porous, biodegradable polymer matrix which is compatible with growth of chondrocytes from the periosteum surrounding the artificial space.

In certain preferred embodiments, the artificial space is created between between tissue layers of an organ, such as between mesenchymal portion of the soft tissue and an adjacent epithelium or compact mesenchymal layer, e.g., the tissue is selected from the group consisting of liver, pancreas, kidney, muscle, spleen, teeth, dentin, mucosa and bone.

In certain other embodiments, the artificial space is created in cardiac tissue.

In still other embodiments, the subject method involves creating an artificial environment in a pre-existing bodily cavity, such as in the pericardial, peritoneal, pleural, synovial, lymph or cerebrospinal cavities/spaces.

The subject method can include the further step of harvesting the pluripotent cells, or tissue derived therefrom, from the artificial space, e.g., to be banked or reimplanted in the animal.

In certain embodiments, the artificial space is created with retractor having a fluid-operated portion, such as a balloon or bladder, to retract a portion of the soft tissue.

In certain preferred embodiments, the area in which the artificial space is to be created is treated with an agent to partially degrade the connective tissue at the site, freeing cells to promote formation of the space and/or promote migration of cells into the space. For example, the area can be treated with an agent is selected from the group consisting of trypsin, chymotrypsin, collagenase, elastase, hyaluronidase, pronase and chondroitinase.

In certain preferred embodiments, the matrix used in the artificial space is a biodegradable matrix, such as a porous, biodegradable polymer. The matrix can include appropriate nutrients for promoting growth of the infiltrating cells. The matrix can also include one or more growth factors for promoting growth of the infiltrating cells. It may also include chemotactic substance for promoting migration of progentior cells into said artificial space. In certain preferred embodiments, the subject matrix includes one or more fibroblast growth factors (FGF) and one or more transforming growth factors, and in even more preferred embodiments, includes basic FGF (bFGF) and TGF-β1 or TGF-β2.

In certain embodiments, such as where the subject method is used to form cartilage or tissue which develops in a relatively avascular environment, it may be desirable to include one or more antiangiogenic agents in the matrix.

In the formation of certain tissues, such as cartilage, it may also be advantageous to apply external pressure to the matrix, such as by application of a pressure bandage or inflated air blatter in the proximal to the cavity.

In certain embodiments, the matrix is a material which is a solution at the time of injection, but which solidifies (gains dimensional stability) in situ. However, after solidification, the matrix should still porous enough to permit migration/infiltration of cells from the surrounding tissue. There are many hydrogels which possess these characteristics, including Pluronics™, sodium or calcium alginates, polyethylene glycol polylactic acid copolymers, and Tetronics™.

The matrix can also include one or more extracellular matrix proteins selected from the group consisting of collagen, chondronectin, fibronectin, vitronectin, proteoglycans, and glycoasmine glycans chains.

Another aspect of the invention relates to a kit for promoting generation of tissue in vivo, comprising:
a. a tissue retractor for generating the artificial space;
b. a matrix which is conducive to infiltration by, and growth and/or differentiation of pluripotent cells; and
c. (optionally) an agent to partially degrade the connective tissue at the site, freeing cells to promote formation of the space and/or promote migration of cells into the space.

Yet another aspect of the invention relates to a method of conducting a regenerative medicine business, comprising:
a. marketing a kit, such as described above, and
b. providing instruction to customers purchasing the kit on how to use the kit for generating tissue in vivo.

Still another aspect of the invention relates to a method of conducting a regenerative medicine business, comprising:
a. providing instruction for carrying out the subject method for isolating cells or tissue from a patient; and
b. providing a cell banking services for preserving the isolated cells or tissue.

Another aspect of the invention provides a method for conducting a regenerative medicine business, comprising:
a. providing instruction for carrying out the subject method to isolate cells or tissue from a patient; and
b. providing a services for further processing the isolated cells or tissue, as for example, to expand the cell population or differentiate the cells.

### BRIEF DESCRIPTION OF THE FIGURES

FIG. 1: Micrographs of a rabbit left leg, 4 weeks after generation of an artificial space which was filled with alginate containing TGF-β1 and b-FGF.

FIGS. 2 and 3: Micrographs of a rabbit left leg, 6 weeks after generation of an artificial space which was filled with alginate (containing no TGF-β1 or b-FGF).

FIG. 4: Micrographs of a rabbit left leg, 8 weeks after generation of an artificial space which was filled with alginate containing TGF-β1 and b-FGF.

FIG. 5: Micrographs of a rabbit left leg, 8 weeks after generation of an artificial space which was filled with alginate (containing no TGF-β1 and b-FGF).

### DETAILED DESCRIPTION OF THE INVENTION

I. Overview

The present invention relates to an in vivo method for promoting the growth of autologous tissue and its use to form corrective structures, including tissue that can be explanted to other locations in the animal. In particular, the invention relates to methods ands systems for (a) the site-specific regeneration of tissue, and (b) the synthesis of neo-tissue for transplantation. This method of the present invention, termed “in vivo bioreactors”, utilizes the patient's own body as the cell source, the scaffold and the drug delivery vehicle. In certain embodiments, the subject approach includes the steps of:
a. creating of a pocket or sac or pouch adjacent to a viable area in the tissue type of interest, e.g., a pocket around the periosteum in the case of bone or an artificial space in a mesenchymal portion of a soft tissue;
b. (optionally) contacting the pocket with an agent, such as an enzyme, that digests extracellular matrix in the surrounding tissue to release cells into the pocket;
c. introducing into the pocket agents or biomaterials, such as growth factors, that promote infiltration by, and growth and/or differentiation of pluripotent cells (stem or progenitor cells) in the pocket.
   The subject method can also be carried out by creating an artificial environment in a pre-existing bodily cavity, such as in the pericardial, peritoneal, pleural, synovial, lymph or cerebrospinal cavities/spaces.

Progenitor cells can be harvested from the space, or alternatively, the cells can be casused to mature to a cell or tissue phenotype of the desired functional and histological end-point, then harvested. Cells/tissue isolated by the subject method can be further manipulated ex vivo, e.g. further expanded or differentiated. The cells/tissue can be banked, e.g, cryogenically preserved, or used for transplantation.

For instance, in certain preferred embodiments, the subject method can be used for promoting generation of cartilage or bone tissue. In such embodiments, the method includes creating an artificial space in or adjacent periosteum tissue of an animal, and then introducing into the artificial space a porous, biodegradable polymer matrix which is compatible with growth of chondrocytes from the periosteum surrounding the artificial space.

In other embodiments, the artificial space in created at a dermal, subdermal and/or intradermal site. Such embodiments can be useful to promote migration of stems from skin or muscle (such as msucle satellite cells) into the artificial space.

In certain embodiments, exogenous cells can be introduced into the artificial space. For instance, the introduced cells can be cells which naturally, or by genetic engineering, produce factors which promote growth or maintenance of stem cells or the progeny thereof which infiltrate the site, and/or aid in the healing process.

In yet other preferred embodiments, the subject methods is used for promoting generation of soft tissue, or precursor cells for soft tissue, comprising the steps of creating an artificial space in a mesenchymal portion of a soft tissue of an animal, and introducing into the artificial space a matrix which is conducive to infiltration by, and growth and/or differentiation of pluripotent cells from the mesenchymal tissue surrounding the artificial space.

There are several advantages to the subject method. For instance, the method uses the patient's own body as the scaffold and bioreactor, thus maximizing the role and impact of the healing process in defining the micro-environment. It uses the patients own cells to engineer/regenerate a tissue mass, thus eliminating the need for harvesting and in vitro culturing of cells. Since the patient's own body and cells will be used to engineer the tissue, immune rejection in a not a issue. It employs a concept of maximizing the role of the body in the healing/regeneration process by minimizing the intervention and hence can be readily adapted to minimally invasive surgical methodologies.

The tissue precursor cells can include any of the following: epidermal cells, chondrocytes and other cells that form cartilage, macrophages, dermal cells, muscle cells, hair follicles, fibroblasts, organ cells, osteoblasts and other cells that form bone, endothelial cells, mucosal cells, pleural cells, ear canal cells, tympanic membrane cells, peritoneal cells, Schwann cells, corneal epithelial cells, gingiva cells, neural cells, neural stem cells such as central nervous system (CNS) stem cells, e.g., spinal cord or brain stem cells, as well as autonomic nervous system (ANS) stem cells, e.g., post-ganglionic stem cells from the small intestine, bladder, liver, kidney, lung, bladder, and heart, (for engineering sympathetic or parasympathetic nerves and ganglia), tracheal epithelial cells, hepatocytes, pancreatic cells, and cardiac cells. The tissue precursor cells can also be neuroendocrine stem cells.

While having a broad applicability in tissue regeneration, in certain preferred embodiments, the subject method can be used to for the generation of osteochondral, liver, kidney, bladder, pancreatic tissues, skeletal muscle, and cardiac muscle.

The device and method are particularly useful for cosmetic surgery, dental implantology and in cardiac surgery. In cosmetic surgery it can be used for soft tissue enlargement like lips and breasts and for facial bones enlargement. In dental implantology, it can be used for horizontal and vertical augmentation of the alveolar ridge when the pouch is placed beneath the periosteum and for sinus augmentation when the pouch is placed beneath the Schneiderian membrane preceding the placement of dental implants. The subject method can also be used for guided bone regeneration in the jaws as part of dental treatment with dental implants.

II. Definitions

A “stem cell” is a relatively undifferentiated cell that can be induced to proliferate and that can produce progeny that subsequently differentiate into one or more mature cell types, while also retaining one or more cells with parental developmental potential. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype.

“Progenitor cells” have a cellular phenotype that is more primitive (i.e., is at an earlier step along a developmental pathway or progression than is a fully differentiated cell). Often, progenitor cells also have significant or very high proliferative potential. Progenitor cells may give rise to multiple distinct differentiated cell types or to a single differentiated cell type, depending on the developmental pathway and on the environment in which the cells develop and differentiate. Like stem cells, it is possible that cells that begin as progenitor cells might proceed toward a differentiated phenotype, but then “reverse” and re-express the progenitor cell phenotype.

A “tissue” is a collection or aggregation of particular cells embedded within its natural matrix, wherein the natural matrix is produced by the particular living cells.

“Differentiation” refers to the developmental process whereby cells assume a specialized phenotype, i.e., acquire one or more characteristics or functions distinct from other cell types. In most uses, the differentiated phenotype refers to a cell phenotype that is at the mature endpoint in some developmental pathway. In many but not all tissues, the process of differentiation is coupled with exit from the cell cycle-in these cases, the cells lose or greatly restrict their capacity to proliferate when they differentiate.

“Proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

“Regeneration” means regrowth of a cell population, organ or tissue after disease or trauma.

“Enriching” of cells means that the yield (fraction) of cells of one type is increased over the fraction of cells of that type in the starting culture or preparation.

As used herein, a “growth factor” includes any soluble factor that regulates or mediates cell proliferation, cell differentiation, tissue regeneration, cell attraction, wound repair and/or any developmental or proliferative process. The growth factor may be produced by any appropriate means including extraction from natural sources, production through synthetic chemistry, production through the use of recombinant DNA techniques and any other techniques which are known to those of skill in the art. The term growth factor is meant to include any precursors, mutants, derivatives, or other forms thereof which possess similar biological activity(ies), or a subset thereof, to those of the growth factor from which it is derived or otherwise related.

A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the cells at the application site, thereby eliminating problems of phagocytosis or cellular death and enhancing new cell growth at the application site. The hydrogels are also biocompatible, e.g., not toxic, to cells suspended in the hydrogel.

The term “channel” refers to a hole of constant or systematically varied cross-sectional area through a sheet of material approximately 50-500 μm thick; with a defined cross-sectional geometry, which may be rectangular, ovoid, circular, or one of these geometries with an imposed finer feature, such as scallops of cell dimension or smaller; defined surface chemistry; and defined dimensions, typically in the range of 75-1000 μm across, with dimensions optimized for each individual tissue or organ type (e.g., preferred channel dimensions for liver in rectangular or ovoid channels is 100-200 μm across one axis with at least 100 μm across on the other axis; embryonic stem cells prefer channels with dimensions between 200 and 1200 μm). Features of the channels are designed to achieve an effect on cell behavior, such as cell organization. The cell behavior does not occur simply because there is an arbitrary hole; the channel is designed to induce cells to organize in the channel to form tissue, either in solid form with blood vessels integrated therein, or in aggregate or spheroidal form. Induction of structure may occur under static conditions (no perfusion) or fluid may be perfused through the channels during morphogenesis to aid formation of histotypical structure, depending on the tissue. One can independently control both the perfusion rate through the array and the nutrient/metabolite/test compound concentrations on each side of the channels by any means.

III. Exemplary Embodiments

A. Tissue Retraction

In preferred embodiments, a tissue retractor is used to generate the artificial space. The retractor selectively moves appropriate tissue out of the way form the space abutting a mesenchymal portion of the tissue or the space in the periosteum. For instance, examples of retractors useful in the methods of the present invention include a fluid-operated portion such as a balloon or bladder to retract tissue, not merely to work in or dilate an existing opening, as for example an angioscope does. The fluid-filled portion of the retractor is flexible and, thus, there are no sharp edges that might injure tissue being moved by the retractor. The soft material of the fluid-filled portion, to an extent desired, conforms to the tissue confines, and the exact pressure can be monitored so as not to damage tissue.

A fluid operated retractor for use in surgery. The retractor has a portion which is expandable upon the introduction of fluid under pressure. The expandable portion is made of a material strong enough, and is inflated to enough pressure, to spread adjoining tissues within the body. In the case of use with tissue such as the periosteum, the expandable portion preferably has sufficient rigidity such that it does deform during the expansion process, e.g., have edges which “leak out” from the site to be expanded.

The bladder can be pressurized with air or with water or another fluid. The fluid used in the bladder must be safe if it accidentally escapes into the body. Thus, besides air, such other fluids as dextrose water, normal saline, CO₂, and N₂ are safe. The pressure in the bladder can be monitored and regulated to keep the force exerted by the retractor at a safe level for the tissue to prevent tissue necrosis. The retractor can exert a pressure on the tissues of as high as the mean diastolic pressure of 100 mm of mercury, or higher for shorter periods of time, while still being safely controlled. Typical inflatable devices such as angioscopes may not be suitable unless adapted to have the strength to hold enough fluid pressure. The bladder may be of such materials such as Kevlar or Mylar which may be reinforced with stainless steel, nylon, or other fiber to prevent puncturing and to provide structural shape and support as desired. Such materials are strong enough to hold the necessary fluid pressure of about several pounds or up to about 500 mg Hg or more and exert the needed force on the tissue to be moved.

In certain embodiments, stents and other barriers can be used to help hold the shape or volume of the expanded area.

In some instances, particularly where the artificial space abuts bone, ultrasonic or other cutting or ablative devices can be used to remove surrounding tissue to permit the expansion of the artificial space.

B. Matrices

In certain embodiments, the artificial space is infused with a matrix which is conducive to infiltration by, and growth and/or differentiation of pluripotent cells from the tissue surrounding the artificial space. Suitable matrices have the appropriate chemical and structural attributes to allow the infiltration, proliferation and differentiation of migrating progenitor cells.

In certain embodiments, the matrices are formed of synthetic, biodegradable, biocompatible polymers. The term “bioerodible”, or “biodegradable”, as used herein refers to materials which are enzymatically or chemically degraded in vivo into simpler chemical species. “Biocompatible” refers to materials which do not elicit a strong immunological reaction against the material nor are toxic, and which degrade into non-toxic, non-immunogenic chemical species which are removed from the body by excretion or metabolism.

The organization of the tissue may be regulated by the microstructure of the matrix. Specific pore sizes and structures may be utilized to control the pattern and extent of tissue ingrowth from the host, as well as the organization of the implanted cells. The surface geometry and chemistry of the matrix may be regulated to control the adhesion, organization, and function of implanted cells or host cells. In certain preferred embodiments, the matrix is formed of polymers having a fibrous structure which has sufficient interstitial spacing to allow for free diffusion of nutrients and gases to cells attached to the matrix surface until vascularization and engraftment of new tissue occurs. The interstitial spacing is typically in the range of 50 to 300 microns. As used herein, “fibrous” includes one or more fibers that is entwined with itself, multiple fibers in a woven or non-woven mesh, and sponge like devices.

The support structure is also biocompatible (e.g., not toxic to the infiltrating cells) and, in some cases, the support structure can be biodegradable. The support structure can be shaped either before or after insertion into the artificial space.

In some cases, it is desirable that the support structure be flexible and/or compressible and resilient. In particular, in these cases, the support structure can be deformed as it is implanted, allowing implantation through a small opening in the patient or through a cannula or instrument inserted into a small opening in the patient. After implantation, the support structure expands into its desired shape and orientation.

In certain embodiments, the matrix is a polymer. Examples of polymers which can be used include natural and synthetic polymers, although synthetic polymers are preferred for reproducibility and controlled release kinetics. Synthetic polymers that can be used include bioerodible polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyamino acids, polyortho esters, polyacetals, degradable polycyanoacrylates and degradable polyurethanes. Examples of natural polymers include proteins such as albumin, collagen, fibrin, and synthetic polyamino acids, and polysaccharides such as alginate, heparin, glycosaminoglycans (such as hyaluronic acid, chondroitin, chondroitin sulfate, dermatan sulfate, heparin, heparan sulfate, keratosulfate, keratopolysulfate and the like), and other naturally occurring biodegradable polymers of sugar units.

In certain embodiments, the matrix is a composite, e.g., of naturally and non-naturally occurring polymers. To illustrate, the matrix can be a composite of fibrin and artificial polymers.

PLA, PGA and PLA/PGA copolymers are particularly useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture”, Chemistry and Industry, 905 (1970).

In certain embodiments, the matrix is a hydrogel. Examples of different hydrogels suitable for practicing this invention, include, but are not limited to: (1) temperature dependent hydrogels that solidify or set at body temperature, e.g., Pluronics™; (2) hydrogels cross-linked by ions, e.g., sodium alginate; (3) hydrogels set by exposure to either visible or ultraviolet light, e.g., polyethylene glycol polylactic acid copolymers with acrylate end groups; and (4) hydrogels that are set or solidified upon a change in pH, e.g., tetronics™.

In certain embodiments, the subject matrix is a photo- or radiation curable polymer. An exemplary photocurable glycosaminoglycan is described in U.S. Pat. No. 5,763,504. In other embodiments, the subject matrix is a chemically curable polymer.

Other examples of materials that can be used to form these different hydrogels include polysaccharides such as alginate, polyphosphazenes, and polyacrylates, which are cross-linked ionically, or block copolymers such as PLURONICS™ (also known as POLOXAMERS™), which are poly(oxyethylene)-poly(oxypropylene) block polymers solidified by changes in temperature, or TETRONICS™ (also known as POLOXAMINES™), which are poly(oxyethylene)-poly(oxypropylene) block polymers of ethylene diamine solidified by changes in pH.

In still other embodiments, the matrix is an ionic hydrogel. Ionic polysaccharides, such as alginates or chitosan, can be used. In one example, the hydrogel is produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix.

All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

In some embodiments, attachment of the cells to the polymer is enhanced by coating the polymers with compounds such as basement membrane components, agar, agarose, gelatin, gum arabic, collagens types I, II, III, IV, and V, fibronectin, laminin, glycosaminoglycans, polyvinyl alcohol, mixtures thereof, and other hydrophilic and peptide attachment materials known to those skilled in the art of cell culture. A preferred material for coating the polymeric matrix is polyvinyl alcohol or collagen.

To promote proliferation and function of the infilitrating the cells, the matrix can additionally contain appropriate nutrients (e.g., serum, salts such as calcium chloride, ascorbic acid, and amino acids) and growth factors (infra).

The matrix may include attachment factors, such as fibronectin, RGD polypeptide, and the like, as well as their analogs, recombinant forms, bioequivalent variants, copolymers or combinations thereof.

Attachment and/or growth factors can be delivered to the site via the shield and spacers. The shields and spacers can be impregnated with these factors during their manufacture, such as during polymerization, or added after manufacture, such as by bonding or crosslinking. The factors may also be encapsulated or similarly treated for their slow release into the site. The shields and spacers can also deliver or fasten to the site a matrix impregnated with attachment and growth factors, such as biodegradable sponges, mesh, fibrin clot, collagen gel, cartilage or other types of biological scaffolding materials made of collagen, hyaluronic acid, polyglycolic acid, polylactic acid, isolated periosteal cells, polydioxane, polyester, alginate, and the like, as well as their analogs or combinations thereof. The matrix can in turn be covered by the membrane described above.

C. Digestion of Extracellular Matrix

According to a further embodiment of the invention, the defect site is treated, preferably prior to implantation, to degrade the connective tissue and extracellular matrix and/or release progenitor cells in the vicinity of the site of the defect, freeing cells (e.g., stromal cells) from that area to migrate into the scaffold of the implant. When enzymes are used to treat the defect site, such enzymes include but are not limited to trypsin, chymotrypsin, collagenase, elastase, and/or hyaluronidase, Dnase, pronase, chondroitinase, etc.

D. Growth Factors

In some embodiments it may be desirable to add bioactive molecules to the cells. A variety of bioactive molecules can be delivered using the matrices described herein. These are referred to generically herein as “factors” or “bioactive factors”.

Bioactive compounds suitable for use in accordance with the present invention include growth factors such as basic fibroblast growth factor (bFGF, or FGF-2), acid fibroblast growth factor (aFGF), epidermal growth factor (EGF), heparin binding growth factor (HBGF), fibroblast growth factor (FGF), vascular endothelium growth factor (VEGF), transforming growth factor (including TGF-α, TGF-β, and bone morphogenic proteins such as BMP-2, -3, -4, -7), Wnts, hedgehogs (including sonic, indian and desert hedgehogs), transforming growth factor-α (TGF-α), noggin, activins, inhibins, insulin-like growth factor (such as IGF-I and IGF-II), growth and differentiation factors 5, 6, or 7 (GDF 5, 6, 7), leukemia inhibitory factor (LIF/HILDA/DIA), Wnt proteins, platelet-derived growth factors (PDGF), vitronectin (VN), laminin (LN), bone sialoprotein (BSP), and osteopontin (OPN), parathyroid hormone related polypeptide (PTHrP), and the like.

Bioactive molecules can be incorporated into the matrix and released over time by diffusion and/or degradation of the matrix, or they can be suspended with the cell suspension. In other embodiments, the bioactive molecules can be provided in the form of microspheres or, as appropriate, produced by exogenous cells which are included in or near the artificial site.

In certain embodiments, instead of a growth factor, antibodies against the growth factor receptor which induce receptor-mediated signal transduction can be used. Likewise, small molecules which agonize receptor activity, e.g., in a ligand-dependent or independent manner, can be used.

In certain embodiments, the subject method employs agonists of Notch function, as described in U.S. Pat. No. 6,149,902. Notch agonists include polypeptides such as Delta and Serrate, antibodies against Notch that induce signal transduction, as well as small molecules which induce Notch-dependent signaling.

In certain embodiments, inhibitors of enzymes which effect proliferation or differentiation of stem/progenitor cells can be used to regulate the infiltrating cells. For example, members of the Kuzbanian metalloprotease family are involved in growth factor response by cells. Agents which inhibit or potentiate the metalloprotease activity can be used to regulate the rate of proliferation or differentiation.

Steroidal anti-inflammatories can be used to decrease inflammation to the implanted matrix, thereby decreasing the amount of fibroblast tissue growing into the artificial space.

These factors are known to those skilled in the art and are available commercially or described in the literature. In vivo dosages are calculated based on in vitro release studies in cell culture; an effective dosage is that dosage which increases cell proliferation or survival as compared with controls, as described in more detail in the following examples.

E. Anti-Angiogenic Factors

In certain embodiments, such as where the subject method is used to form cartilage or tissue which develops in a relatively avascular environment, it may be desirable to include one or more antiangiogenic agents in the matrix.

The term “antiangiogenic agent” refers to a composition that is capable of reducing the formation or growth of blood vessels. Examples of antiangiogenic agents include, but are not limited to, endostatin protein, angiostatin protein, TNP-470, angiozyme, anti-VEGF, benefin, BMS275291, bryostatin-I (SC339555), CAI, CM101, combretastatin, dexrazoxane (ICRF187), DMXAA, EMD 121974, flavopiridol, GTE, IM862, interferon-α, interlukin-12, inhibitors of matrix metalloproteinases such as marimastat, metaret, metastat, MMI-270, neovastat, octreotide (somatostatin), paclitaxel (taxol), purlytin, PTK787, squalarnine, suradista (FCE26644), SU101, SU5416, SU6668, tamoxifen (nolvadex), tetrathiomolybdate, thalidomide, vitaxin and xeloda (capecitabine), cycloogenase, platelet factor 4 (PF-4), an N-terminally truncated proteolytically cleaved PF-4 fragment, a 16 kDa N-terminal fragment of human prolactin, smaller protein fragments of fibronectin, murine epidermal growth factor, and thrombospondin.

Additionally, as used herein, the term “angiostatinprotein” refers to a kringle region fragment of a plasminogen molecule that has antiangiogenic activity in vivo. Examples of angiostatin proteins may be found in U.S. Pat. No. 5,837,682 and U.S. Pat. No. 5,854,221. Plasminogen contains five kringle region fragments, denoted kringles 1-5, as well as inter-kringle regions. It is to be understood that the term “angiostatin protein” refers to any single kringle region, any combination of kringle regions, or any kringle regions in addition to any inter-kringle regions that retain antiangiogenic activity in vivo. In a preferred embodiment, angiostatin protein is approximately kringle regions 1-3, kringle regions 1 5, kringle regions 1-4 or kringle regions 1-5 of human plasminogen.

The terms “endostatin protein” and “angiostatin protein” also include shortened proteins wherein one or more amino acid is removed from either or both ends of an endostatin protein or an angiostatin protein, respectively, or from an internal region of either protein, yet the proteins retains angiogenesis inhibiting activity in vivo. The terms “endostatinprotein” and “angiostatin protein” also include lengthened proteins or peptides wherein one or more amino acids is added to either or both ends of an endostatin protein or an angiostatin protein, respectively, or to an internal location, yet the proteins retain angiogenesis inhibiting activity in vivo.

Also included within the terms “angiostatin protein” and “endostatin protein” are angiostatin protein and endostatin protein derivatives. An angiostatin protein derivative includes a protein having the amino acid sequence of a kringle region fragment of a plasminogen that has antiangiogenic activity. An angiostatin protein also includes a peptide having a sequence corresponding to an antiangiogenic angiostatin fragment of a kringle region fragment of a plasminogen. An “antiangiogenic angiostatin fragment” is defined to be a peptide whose amino acid sequence corresponds to a subsequence of a kringle region fragment of a plasminogen, referred to as an “antiangiogenic angiostatin subsequence”.

The antiangiogenic agent can also be a VEGF receptor tyrosine kinase inhibitor. Exemplary antiangiogenic agents of that class include:
4-(4-bromo-2-fluoro-5-hydroxyanilino)-6,7-dimethoxyquinazoline;
4-(4-chloro-2-fluoro-5-hydroxyanilino)-6-methoxy-7-(2-thiomorpholinoethoxy) quinazoline;
6,7-dimethoxy-4-(3-hydroxy-4-methylphenoxy)quinazoline;
4-(4-chloro-2-fluoro-5-hydroxyanilino)-6-methoxy-7-(3-morpholinopropoxy)qui nazoline;
4-(2-fluoro-5-hydroxy-4-methylanilino)-7-(2-hydroxyethoxy)-6-methoxyquinazo line;
4-(4-chloro-2-fluoro-5-hydroxyanilino)-6-methoxy-7-(2-(4-methylpiperazin-1-yl)ethoxy)quinazoline;
4-(2-fluoro-5-hydroxy-4-methylanilno)-7-(2-methoxyethoxy)qunazoline;
4-(2-fluoro-5-hydroxy-4-ethylanilino)-6-methoxy-7-(2-(methylsulphinyl)etho xy)quinazoline;
4-(4-chloro-2-fluoro-5-hydroxyanilino)-6-methoxy-7-(2-methoxyethoxy)quinazo line;
4-(4-chloro-2-fluoro-5-hydroxyanilino)-6,7-dimethoxyquinazoline;
4-(2-fluoro-5-hydroxy-4-methylanilino)-6,7-dimethoxyquinazoline;
4-(2-fluoro-5-hydroxy-4-methylanilino)-6-methoxy-7-(2-methoxyethoxy)quinazo line;
7-(2-acetoxyethoxy)-4-(2-fluoro-5-hydroxy-4-methylanilino)-6-methoxyquinazo line;
4-(4-chloro-2-fluoro-5-hydroxyanilino)-6-methoxy-7-(2-morpholinoethoxy)quin azoline;
4-(4-chloro-2-fluoro-5-hydroxyanilino)-6-methoxy-7-(2-piperidinoethoxy)quin azoline;
4-(4-chloro-2-fluoro-5-hydroxyanilino)-6-methoxy-7-(2-(pyrrolidin-1-yl)etho xy)quinazoline;
4-(2-fluoro-5-hydroxy-4-methylanilino)-7-(2-methoxyethylamino)quinazoline;
4-(4-chloro-2-fluoro-5-hydroxyanilino)-6-methoxy-7-(2-cyclopentyloxyethoxy) quinazoline;
4-(2-fluoro-5-hydroxy-4-methylanilino)-6-methoxy-7-(2-methylthioethoxy)quin azoline;
4-(2,4-difluoro-5-hydroxyanilino)-6,7-dimethoxyquinazoline;
4-(2,4-difluoro-5-hydroxyanilino)-6-methoxy-7-(2-methoxyethoxy)quinazoline;
4-(2-fluoro-5-hydroxy-4-methylanilino)-6-methoxy-7-(3-morpholinopropoxy)qui nazoline;
4-(2-fluoro-5-hydroxy-4-methylanilino)-7-methoxyacetamidoquinazoline;
4-(4-bromo-2,6-difluoroanilino)-6-methoxy-7-(3-morpholinopropoxy)quinazoline, and salts thereof especially the hydrochloride salts thereof.

F. Periosteum Bioreactors

The role of periosteum (perichondrium) in the development of skeletal tissue in a developing embryo is well established. See Developmental Anatomy, 6^{th} Edition, L. B. Arey, (Saunders) (1954); and Yoo et al. Clin. Orthop., Suppl. 355, S73-81 (1998). The cambium layer contains chondrogenic cells that become the source of the formation and evolution of the limb bud in utero. Thus it is logical that the periosteum can and should play an active role in the healing and regeneration of osseous and chondral tissue. However, the utility of periosteum in the repair and regeneration of osseous and chondral defects in adults has been barely explored. The only surgical procedure to date involving the periosteum is in the repair of defects in the articular surface, using a periosteal flap in conjunction with enzymatic digestion and cell transplantation in repair of the articular surface. This process, also known as the Genzyme-Carticel process has been a moderate success.

O'Driscoll and co-workers have pioneered the effort in understanding and harnessing the potential of the Periosteum. They have demonstrated using a “organ culture model” that under aerobic conditions using standard culture medium supplemented with fetal calf serum and TGF-beta, a cartilaginous tissue matrix can be obtained from a harvested periosteum in vitro^{2,8}.

O'Driscoll et al. Clin. Orthop. Suppl. 367, S186-203 (1999); and O'Driscoll et. al. J. Bone Joint Surg. Am., 76(7), 1042-1051 (1994). They have also shown that two factors namely, the donor site (Gallay et. al., J. Orthop. Res., 12(4), 515-525 (1994)) and maintaining the viability of the periosteum after explantation are critical in achieving chondrogenesis in vitro (O'Driscoll et. al. Cell Transplant., 8(6), 611-616 (1999)).

Notwithstanding, the advantages of the periosteum approach as explored currently i.e., the periosteum serves as (a) the source of cells, and (b) the source of bioactive agents for defining the local environment, it has two serious drawbacks. They are (a) obtaining and maintaining the viability of the periosteum, and (b) the ex vivo culturing of the periosteum to a well-defined end point.

The “in vivo bioreactor” paradigm not only serves to address the issues raised by O'Driscoll but also solves the issue of periosteum viability and ex vivo manipulation.

In one embodiment, the subject method includes the following steps in the generation of cartilaginous tissue using the “in vivo bioreactor” approach.
1. Creation of a pocket between the cambium layer of the periosteum and the bone using a combination of techniques similar to balloon angioplasty and bone debriment.
2. Filling of the pocket with a gel containing growth factors with or with out enzymatic digestion of the cambium.
3. Maturation of the pocket by the infiltration of cartilaginous tissue.
4. Biopsy of the tissue if necessary.
5. Harvesting the periosteum-cartilaginous tissue
6. Transplantation onto either the articular surface or bony site.

Thus the “in vivo bioreactor” will allow for the manipulation of the periosteum while it is still attached to the bone. This will ensure the viability of the periosteum throughout the duration of manipulation. The creation of a pocket between the periosteum and bone will allow for the alteration of the environment with biomaterials (scaffolds) and growth factors while preserving the natural milieu and taking advantage of the natural healing process. One can also potentially exploit the positive effects of the mechanical deformation of the periosteum in chondrogenesis. Furthermore, the creation of a pocket around the periosteum alleviates the need the ex vivo culturing of the periosteum and/or supplementation using cells cultured ex vivo. This is a big advantage from a clinical, time and FDA standpoint Finally, the generation of a tissue in vivo as described herein offers the opportunity to grow both cartilaginous and osseous tissue under identical conditions.

### IV. EXAMPLE

A. Preparation of Alginate Gels

Sodium alginate solutions of 1%, 2%, 2.5% and 3% (w/v) were made up in 30 mM Hepes containing 150 mM NaCl and 10 mM KCl. Gelation of these solutions was triggered by the addition of an equal volume of a solution containing either 200 or 300 mM CaSO₄, or 50, 75, 100, 150 or 300 mM CaCl₂ in 10 mM Hepes and containing 150 mM NaCl and 10 mM-KCl. All solutions were sterilised by autoclaving and were mixed utilizing a sterile homemade Y-piece. Gelation time was determined visually. Gels were also inspected for homogeneity in appearance including the presence of calcium salt precipitates.

Ionically crosslinked alginate hydrogels were prepared from four sodium alginates, the composition, intrinsic viscosity and molecular weights of which are detailed in Table 1. It should be noted that two of the alginates had a relatively low percentage of guluronic acid (40%) and will be referred to as M1 and M2. The two alginates with a relatively high guluronic acid content (65-75%) will be referred to as G1 and G2 (NB. The alginate we use in the in vivo formulation is G2).

The gelation of the sodium alginates as triggered by the addition of divalent ions was investigated using CaSO₄ or CaCl₂ as a source of calcium ions. A homemade Y-piece was developed to mix the two solutions and allow a more uniform distribution within the final product. In contrast if a diffusional setting method is used, depletion of alginate is observed in the internal non-gelled part of the gelling body as the alginate molecules in this part diffuse outwards towards the zero activity region in the sharp gelling zone that is created.

Gelation could be induced by addition of 200 or 300 mM CaSO₄ to a 2% (w/v) solution of the sodium alginates. However a period of several hours was required for gelation to reach completion. Furthermore, in each instance, CaSO₄ precipitates were apparent throughout the gel, particularly when 300 mM CaSO₄ was utilized. The presence of precipitates decreased the gel homogeneity and may also negatively impact on the diffusion and viability of cells within the gel matrix.

The use of CaCl₂ to achieve a more rapid cross-linking of the sodium alginates without the formation of precipitates was investigated. Rapid gelation (<1 min) was induced in 1% and 2% (w/v) solutions of M1, M2, G1 and G2 utilizing solutions containing 50, 75, or 100 mM CaCl₂. The gels formed with 1% (w/v) sodium alginate solutions or with 50 mM CaCl₂, consistently formed gels with unacceptably weak mechanical properties and were not further investigated here. In contrast, gels formed utilizing 2% (w/v) alginate and a higher concentration of calcium ions (75 and 100 mM) were mechanically more stable as determined visually.

To facilitate cell diffusion into the gel matrix for in vitro studies, higher gel porosity is favored. This can be achieved by utilizing alginates rich in guluronic acid residues which contain long blocks of guluronic acid residues and where the length of flexible elastic segments is minimised allowing a more stiff open and static network to be formed. For this reason, increasing the guluronic acid content of alginate up to 70% has also been observed to lead to enhanced mechanical rigidity and higher moduli for the gels. The likely more porous structure afforded by gels formed from G1 and G2 was therefore deemed more suitable for tissue engineering applications than gel formation utilizing M1 and M2. Of the gels formed with G1 and G2, G2 consistently produced more homogeneous gels due to the higher molecular weight and intrinsic viscosity of G2 relative to G1 (see Table 1). Homogeneity in all gel samples was decreased in the absence of non-gelling ions (data not shown).

The gelation potential of G2 was further investigated by preparing gels formed from 2%, 3% or 4% (w/v) solutions of G2 and 75, 100, 150 or 300 mM CaCl₂ solutions. In each instance gelation was very rapid (<1 min) and gels appeared homogeneous when visually inspected. Gels formed by combining 3% or 4% (w/v) solutions of G2 with 100, 150 and 300 mM CaCl₂ solutions utilizing the Y-piece produced small discretely defined hard pellets. In contrast when gelation was induced in the 2% (w/v) G2 solution by addition of 75 mM CaCl₂, the gel form was such that it could be easily shaped and molded and thus suitable for potential in vivo applications requiring injectable delivery.

TABLE 1
The composition, intrinsic viscosity, molecular weight and
supplier for the alginates employed

| | | | Intrinsic | | |
|---|---|---|---|---|---|
| | | Composition | Viscosity of | | |
| Alginate | | (Frequency of | 1% Solution | Molecular | |
| (Sodium Salt) | | Guluronic Acid) | (mPas) | Weight (Da) | Supplier |
| Macrocystic | M | 0.4 | 20-25 | 12,000-80,000 | Sigma |
| Pyrifera | 1 | | | | |
| Macrocystic | M | 0.4 | 80-200 | 80,000-120,000 | Sigma |
| Pyrifera | 2 | | | | |
| Laminaria | G | 0.65-0.75 | 20-70 | 120,000-150,000 | FMC |
| Hyperborea | 1 | | | | BioPolymer |
| Laminaria | G | 0.65-0.75 | 200-400 | 300,000-350,000 | FMC |
| Hyperborea | 2 | | | | BioPolymer |

B. In Vitro Experiments

An in vitro study of chondrogenesis using an organ culture model developed by O'Driscoll (supra), was performed to determine the timeframe and conditions required for periosteal chondrogenesis.

A further in vitro study of periosteal chondrogenesis has been performed to evaluate an organ culture model using an alginate gel suspension. Briefly, periosteal explants of approximate dimensions 3×3 mm were cultured in an alginate gel suspension and supplemented with either TGF-β1, b-FGF, a combination of the two, or in the absence of growth factors. b-FGF and TGF-β1 were administered at a concentration of 10 ng/ml every two days at each media change for the first week and first two weeks of in vitro culture respectively.

Additionally the cultures were supplemented daily with 50 μg/ml ascorbic acid for the first four weeks of in vitro culture. Results from histological analysis (H & E; and Safranin-O staining) of the explants performed at 1, 3, 6 and 8 week timepoints, were similar to those from the organ culture model utilizing the agarose gel (data not shown). Namely, after a period of 1 week in vitro culture, none of the explants stained positive for glycosaminoglycans using Safranin-O staining. Significant neo-chondrogenesis from the periosteum was not observed for explants cultured in the absence of added growth factors or supplemented with b-FGF alone over the period of 8 weeks. Chondrogenesis was apparent after 3 weeks in vitro culture if exogenous TGF-β1 was administered, and was most pronounced after 8 weeks in vitro culture. As with the previous agarose based organ culture study, neo-chondrogenesis was further enhanced if the in vitro culture was supplemented with the combination of b-FGF and TGF-1. Safranin-O stains of typical periosteal explants after 1, 3, 6 and 8 weeks in vitro culture when b-FGF and TGF-β1 were administered for the first week and first two weeks of in vitro culture were observed. The glycosaminoglycan rich neo-tissue was found to contain collagen type II as characterised using immunohistochemical methods.

These in vitro studies have allowed timepoints for the initial in vivo feasibility study (see below) to be established. Furthermore it is apparent from these studies that incorporation of both b-FGF and TGF-β1 into the gel formulation may enhance periosteal chondrogenesis in vivo.

In order to extend the applicability of the observation that b-FGF can act synergistically with TGF-β1 to enhance periosteum-derived chondrogenesis, a further in vitro study using the organ culture model is currently being performed utilizing bovine periosteal tissue. Neo-chondrogenesis in the periosteal explants will be evaluated by histological analysis (H & E; and Safranin-O staining), immunohistochemical methods and biochemical analysis at 4, 7, 10, 14, 21 and 28 day timepoints.

C. In Vivo Experiments

An initial feasibility study was carried out and involved performing a survival surgery procedure to create an artificial space in the periosteum in the tibia of New Zealand rabbit models in which neo-cartilaginous tissue was regenerated. Initial experiments involved the perfection of the surgical technique. This involved creating an artificial space (of approximate dimensions 1×1 centimeter and 0.5-1 centimeter in depth) in the periosteum in the tibia. It was found that enzymatic digestion of the periosteum was not necessary to partially degrade the connective tissue at the site or to promote formation of the space and/or promote migration of cells into the space. A biodegradable alginate gel, which was compatible with growth of chondrocytes from the periosteum surrounding the space, was introduced into the artificial space.

Using the surgical procedure, implantation of an alginate gel containing growth factors was performed on 12 rabbits. These were sacrificed at time-points of 1, 2, 4, 6, 8 or 12 weeks post surgery and histological analysis of the artificial pocket and the surrounding area was performed. This allows the presence of neo-cartilage in the artificial space to be determined at each of the time-points and the effects of the alginate gel on the surrounding tissue to be evaluated. These studies established the viability of the creation of a periosteal pocket for in vivo generation of neo-tissue. Furthermore it is apparent that there is no visible discomfort to the rabbits post-operatively. The time-points were chosen based On results obtained from a concurrent in vitro studies of chondrogenesis.

Using this procedure, the surgical procedure was performed to create an artificial space in the periosteum in the tibia of both the right and left hind legs of the rabbit models in which neo-tissue could be regenerated. An alginate gel containing no growth factors (control) or containing both b-FGF and TGF-β at a concentration of 10 ng/ml was introduced into the artificial space. The “periosteal pockets” were subsequently sealed with a fibrin glue and the gel found to be retained in place.

Time-points for the in vivo maturation of neo-tissue in the pocket were determined from the in vitro studies of chondrogenesis. The rabbits were sacrificed at 4, 6, 8 and 12 week time-points and the whole tibia decalcified using EDTA.

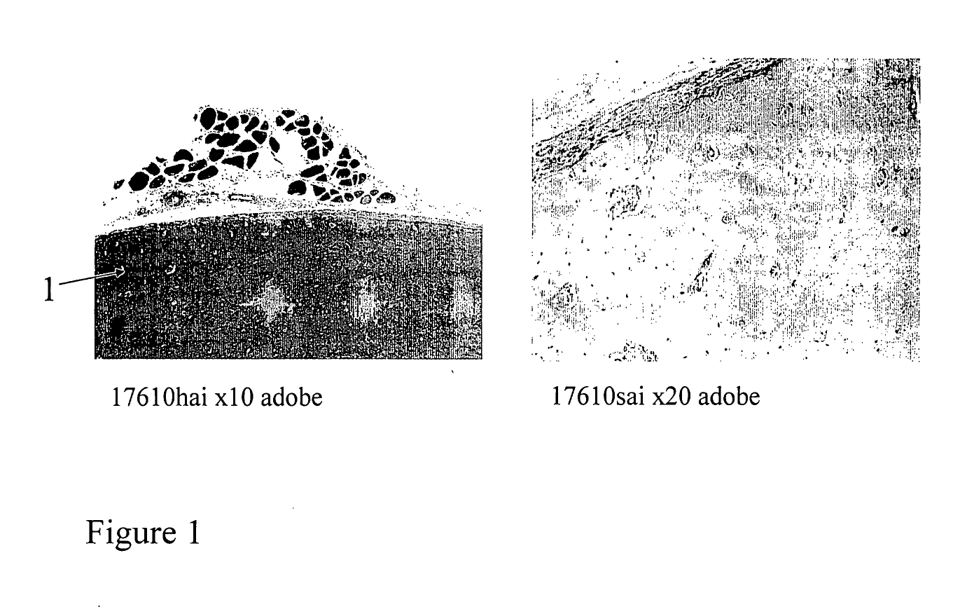

FIG. 1 is a micrograph of a rabbit left leg, 4 weeks after generation of an artificial space which was filled with alginate containing TGF-β1 and b-FGF. 1 is the area of new bone formation that has occurred in the pocket.

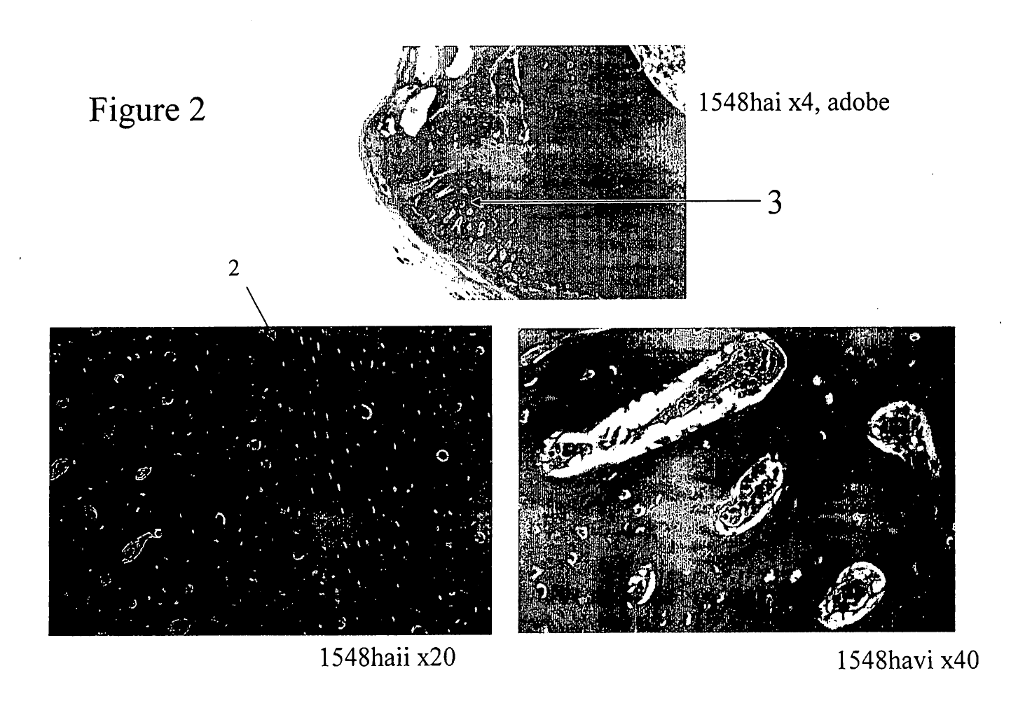

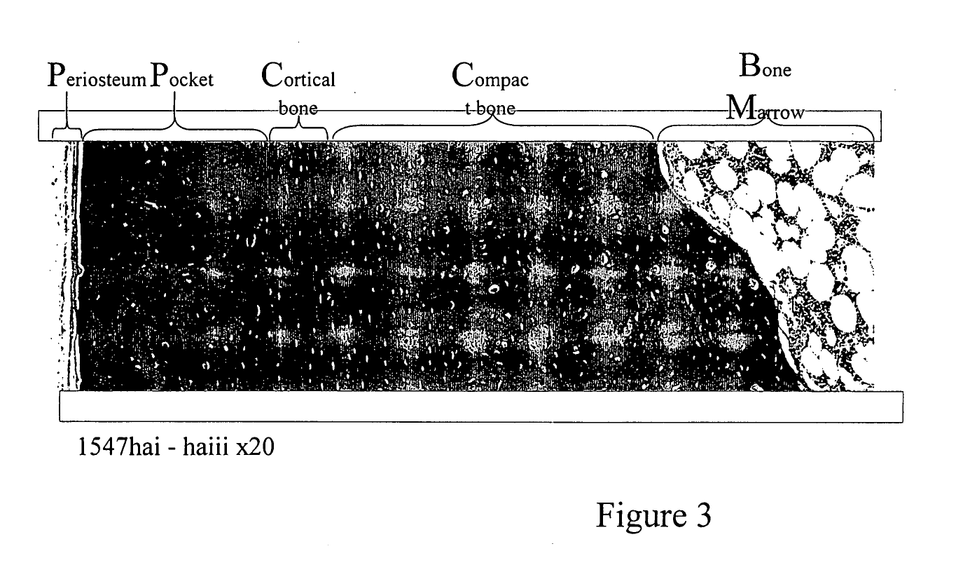

FIG. 2 is a micrograph of a rabbit left leg, 6 weeks after generation of an artificial space which was filled with alginate (containing no TGF-β1 or b-FGF). 3 is the boundary between the artificial space (pocket) and the bone. New bone is to the left. As expected in new bone growth, the area of new bone growth is populated with large blood vessels 4. FIG. 3 shows the cross-section of the bone from edge of bone to medullary cavity.

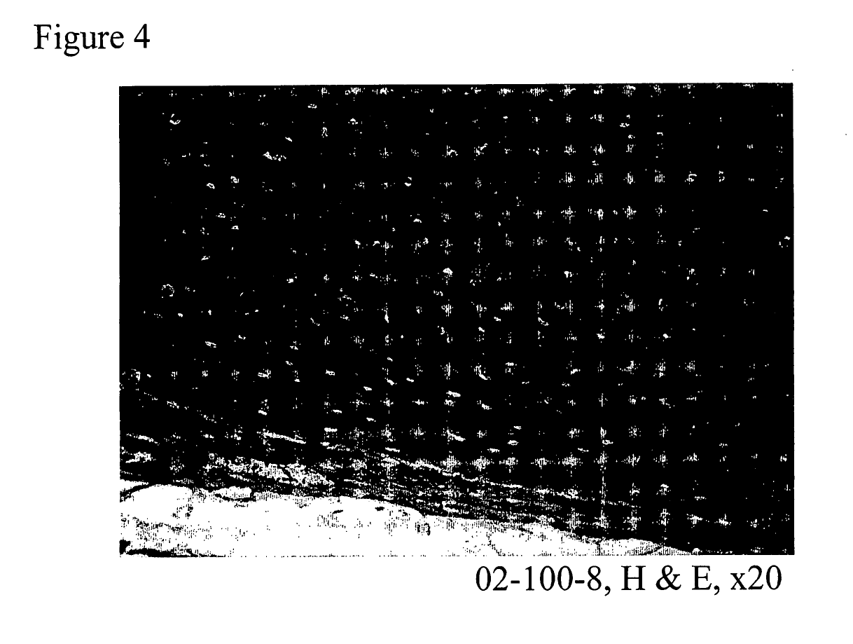

FIG. 4 is a micrograph of a rabbit left leg, 8 weeks after generation of an artificial space which was filled with alginate containing TGF-β1 and b-FGF). The periosteum now looks normal, blood vessels are no longer larger than in normal bone and appearance of bone in general is more mature (stains darker).

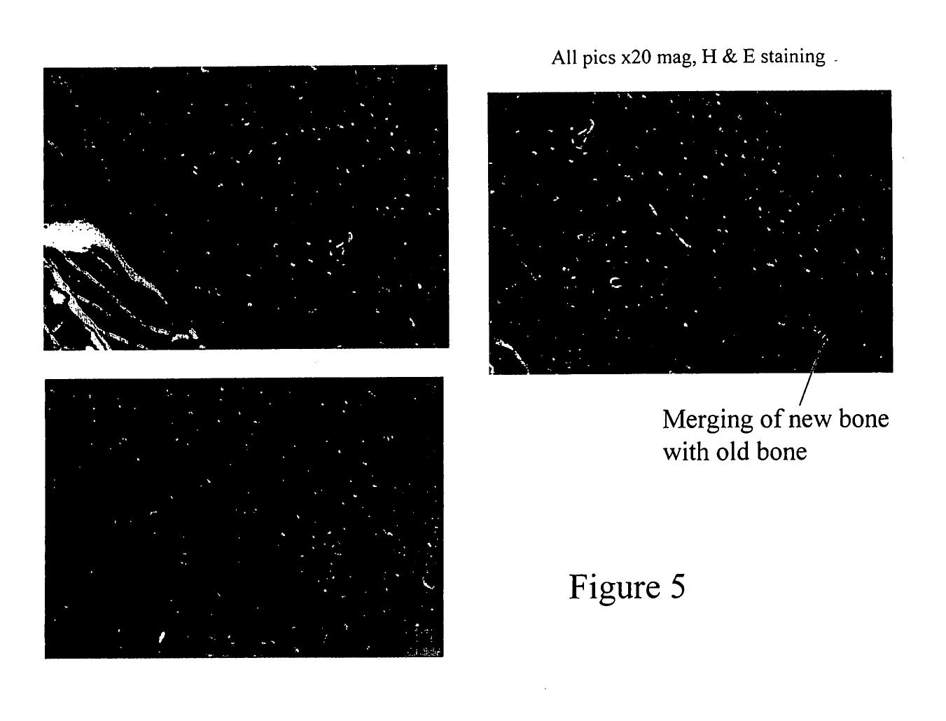

FIG. 5 are micrographs of a rabbit left leg, 8 weeks after generation of an artificial space which was filled with alginate (containing no TGF-β1 and b-FGF). The morphology looks similar to the growth factor treated leg at 8 weeks. As indicated, merging of new bone with old bone was observed.

1. A method for promoting generation of soft tissue, or precursor cells for soft tissue, comprising the steps of:
i. creating an artificial space or environment in an organ or cavity of an animal; and
ii. introducing into the artificial space a matrix which is conducive to infiltration by, and growth and/or differentiation of pluripotent cells from the tissue surrounding the artificial space.

2. The method of claim 1, including the further step of harvesting the pluripotent cells, or tissue derived therefrom, from the artificial space.

3. The method of claim 2, wherein the harvested cells are reimplanted in the animal.

4. The method of claim 1, wherein the artificial space is created in or adjacent periosteum tissue.

5. The method of claim 1, wherein the artificial space is created between a mesenchymal portion of a soft tissue organ and an adjacent epithelium or compact mesenchymal layer of the organ.

6. The method of claim 1, wherein the tissue is selected from the group consisting of liver, pancreas, kidney, muscle, spleen, teeth, dentin, mucosa and bone.

7. The method of claim 1, wherein the artificial space is created with retractor having a fluid-operated portion, such as a balloon or bladder, to retract a portion of the soft tissue.

8. The method of claim 1, wherein the area in which the artificial space is to be created is treated with an agent to partially degrade the connective tissue at the site, freeing cells to promote formation of the space and/or promote migration of cells into the space.

9. The method of claim 7, wherein the agent is selected from the group consisting of trypsin, chymotrypsin, collagenase, elastase, hyaluronidase, pronase and chondroitinase.

10. The method of claim 1, wherein the matrix is a porous, biodegradable polymer.

11. The method of claim 1, wherein the matrix is a solution at the time of injection into the artificial space or environment, but which gains dimensional stability in situ.

12. The method of claim 1, wherein the matrix is a hydrogel.

13. The method of claim 12, wherein the hydrogels is selected from the group consisting of a Pluronics hyrgogel, an alginates, a hydrogel formed from polyethylene glycol polylactic acid copolymers, and a Tetronics hydrogel.

14. The method of claim 1, wherein the matrix includes appropriate nutrients for promoting growth of said infiltrating cells.

15. The method of claim 1, wherein the matrix includes one or more growth factors for promoting growth and/or differentiation of said infiltrating cells.

16. The method of claim 15, wherein said one or more growth factors are selected from the group consisting of basic fibroblast growth factor (bFGF, or FGF-2), acid fibroblast growth factor (aFGF), epidermal growth factor (EGF), heparin binding growth factor (HBGF), fibroblast growth factor (FGF), vascular endothelium growth factor (VEGF), transforming growth factor (including TGF-α, TGF-β, and bone morphogenic proteins such as BMP-2, -3, -4, -7), Wnts, hedgehogs (including sonic, indian and desert hedgehogs), transforming growth factor-(x (TGF-α), noggin, activins, inhibins, insulin-like growth factor (such as IGF-I and IGF-II), growth and differentiation factors 5, 6, or 7 (GDF 5, 6, 7), leukemia inhibitory factor (LIF/HILDA/DIA), Wnt proteins, platelet-derived growth factors (PDGF), vitronectin (VN), laminin (LN), bone sialoprotein (BSP), and osteopontin (OPN), parathyroid hormone related polypeptide (PTHrP), and the like.

17. The method of claim 1, wherein the matrix includes one or more anti-angiogenic agents.

18. The method of claim 1, wherein the matrix includes one or more extracellular matrix proteins selected from the group consisting of collagen, chondronectin, fibronectin, vitronectin, proteoglycans, and glycoasminoglycans chains.

19. The method of claim 1, wherein the matrix includes is a composite of naturally and artificial polymers.

20. The method of claim 1, wherein the biodegradable matrix includes a chemotactic substance for promoting migration of said infiltrating cells into said artificial space.

21. The method of claim 1, wherein the shields and/or spacers are placed in the artificial space.

22. A method for promoting generation of cartilage or bone tissue, comprising the steps of:
i. creating an artificial space in or adjacent periosteum tissue of an animal; and
ii. introducing into the artificial space a porous, biodegradable polymer matrix which is compatible with growth of chondrocytes from the periosteum surrounding the artificial space.

23. The method of claim 22, wherein the biodegradable matrix includes appropriate nutrients for promoting growth of said chondrocytes.

24. The method of claim 22, wherein the biodegradable matrix includes one or more growth factors for promoting growth of said chondrocytes.

25. The method of claim 24, wherein said growth factors are selected from the group consisting of a somatomedin, a hedgehog protein, parathyroid related hormone, a basic fibroblast growth factor, a transforming growth factor-β, a cartilage growth factor, and combinations thereof.

26. The method of claim 25, wherein the biodegradable matrix includes one or more anti-angiogenic agents.

27. The method of claim 22, wherein the artificial space is treated with an agent to partially degrade the connective tissue at the site, freeing cells to promote formation of the space and/or promote migration of cells into the space.

28. The method of claim 27, wherein the agent is selected from the group consisting of trypsin, chymotrypsin, collagenase, elastase, hyaluronidase, pronase and chondroitinase.

29. A kit for promoting generation of tissue in vivo, comprising:
a. a tissue retractor for generating the artificial space;
b. a matrix which is conducive to infiltration by, and growth and/or differentiation of pluripotent cells; and
c. (optionally) an agent to partially degrade the connective tissue at the site, freeing cells to promote formation of the space and/or promote migration of cells into the space.

30. A kit for promoting generation of tissue in vivo, comprising:
a. a matrix precursor(s) capable of forming a dimensionally stable matrix in vivo, which matrix is conducive to infiltration by, and growth and/or differentiation of pluripotent cells, which matrix precursor(s) is in the form of a solution;
b. a TGF-β and b-FGF, admixed with said matrix precursor(s) or in a form which is amenable to mixture with said matrix precursor(s); and
c. instructions (written or pictorial) associated with said kit, said instructions describing the preparation of the matrix precursor(s), TGF-β and b-FGF for injection into an artificial space or environment in vivo.

31. A method of conducting a regenerative medicine business, comprising:
a. marketing a kit of claim 29 or 30, and
b. providing instruction to customers purchasing the kit on how to use the kit for generating tissue in vivo.

32. A method of conducting a regenerative medicine business, comprising:
a. providing instruction for carrying out the method of claim 1 or 22 to isolate cells or tissue from a patient; and
b. providing a cell banking services for preserving the isolated cells or tissue.

33. A method of conducting a regenerative medicine business, comprising:
a. providing instruction for carrying out the method of claim 1 or 22 to isolate cells or tissue from a patient; and
b. providing a services for further processing the isolated cells or tissue, as for example, to expand the cell population or differentiate the cells.
